(19) United States
(12) Patent Application Publication
(10) Pub. No.: US 2010/0248259 A1
(43) Pub. Date: Sep. 30, 2010

(54) NATRIURETIC PEPTIDE/GDF-15 RATIO FOR DIAGNOSIS OF CARDIAC DISORDERS

(76) Inventors: Gerog Hess, Mainz (DE); Andrea Horsch, Mannheim (DE); Dietmar Zdunek, Tutzing (DE)

Correspondence Address: ROCHE DIAGNOSTICS OPERATIONS INC., 9115 Hague Road, Indianapolis, IN, 46250-0457, US

(21) Appl. No.: 12/710,696

(22) Filed: Feb. 23, 2010

Related U.S. Application Data
(63) Continuation of application No. PCT/EP2008/061407, filed on Aug. 29, 2008.

(30) Foreign Application Priority Data
Aug. 30, 2007 (EP) 07115303.5

(51) Int. Cl. G01N 33/53; G01N 33/566; C12M 1/34; G01N 30/96

(52) U.S. Cl. 435/7.1; 436/501; 435/287.2; 422/69

(57) ABSTRACT

The present invention is concerned with methods and devices for medical diagnosis. Specifically, it relates to a method of diagnosing a cardiac disorder, the method including (a) determining the amount of a natriuretic peptide in a sample of the subject, (b) determining the amount of GDF-15 in a sample of the subject, (c) calculating the ratio (natriuretic peptide/GDF-15), and (d) diagnosing if the subject is suffering from a cardiac disorder, based of the ratio calculated in step (c). The method allows determining whether an elevated amount of GDF-15 in a sample of a subject is related to cardiac disorders, in particular myocardial dysfunction and/or heart failure, or if the elevation is related to a different pathological state Moreover, the present invention relates to a diagnostic device and a kit for carrying out the aforementioned method.

### RELATED APPLICATIONS

This application is a continuation of international application PCT/EP2008/061407 filed Aug. 29, 2008 and claims priority to European application EP 07115303.5 filed Aug. 30, 2007.

### FIELD OF THE INVENTION

The present invention is concerned with methods and devices for medical diagnosis. Specifically, it relates to a method of discriminating if an elevated amount of GDF-15 in a sample of a subject is related to cardiac disorders, in particular myocardial dysfunction and/or heart failure, or if the elevation is related to a different pathological state like, for example, a liver, pulmonary or kidney malfunction or even a tumor, said method comprising determining the amounts of GDF-15 and a natriuretic peptide in a sample of said subject and diagnosing, from the ratio of the natriuretic peptide to GDF-15, if the patient is suffering from a cardiovascular disorder. Moreover, the present invention relates to a diagnostic device and a kit for carrying out the aforementioned method.

### BACKGROUND OF THE INVENTION

An aim of modern medicine is to provide personalized or individualized treatment regimens. Those are treatment regimens which take into account a patient's individual needs or risks. Personalized or individual treatment regimens shall be even taken into account for emergency measures where it is required to decide on potential treatment regimens within short periods of time. Heart diseases are the leading cause of morbidity and mortality in the Western hemisphere. The said diseases can remain asymptomatic for long periods of time. However, they may have severe consequences once an acute cardiovascular event, such as myocardial infarction, as a cause of the cardiovascular disease occurs.

Heart failure is a condition that can result from any structural or functional cardiac disorder that impairs the ability of the heart to fill with or pump a sufficient amount of blood throughout the body. Even with the best therapy, heart failure is associated with an annual mortality of about 10%. Heart failure is a chronic disease; it can, inter alia, occur either following an acute cardiovascular event (like myocardial infarction), or it can occur, e.g., as a consequence of inflammatory or degenerative changes in myocardial tissue. Heart failure patients are classified according to the NYHA system in classes I, II, III and IV. A patient having heart failure will not be able to fully restore his health without receiving a therapeutic treatment.

Myocardial dysfunction is a general term, describing several pathological states of the heart muscle (myocard). A myocardial dysfunction may be a temporary pathological state (caused by, e.g., ischemia, toxic substances, alcohol, . . . ), contrary to heart failure. Myocardial dysfunction may disappear after removing the underlying cause. A symptomless myocardial dysfunction may, however, also develop into heart failure (which has to be treated in a therapy). A myocardial dysfunction may, however, also be a heart failure, a chronic heart failure, even a severe chronic heart failure.

Myocardial dysfunction and heart failure often remain undiagnosed, particularly when the condition is considered “mild.” The conventional diagnostic techniques for heart failure are based on the well known vascular volume stress marker NT-proBNP. However, the diagnosis of heart failure under some medical circumstances based on NT-proBNP appears to be incorrect for a significant number of patients but not all (e.g., Beck 2004, Canadian Journal of Cardiology 20: 1245-1248; Tsuchida 2004, Journal of Cardiology, 44:1-11). However, especially patients which suffer from heart failure would urgently need a supportive therapy of the heart failure. On the other hand, as a consequence of an incorrect diagnosis of heart failure, many patients will receive a treatment regimen which is insufficient or which may have even adverse side effects.

The non-prepublished European patent application 07108852.0 of the applicant, filed on May 24, 2007, discloses a method of diagnosing heart failure in a subject exhibiting atrial fibrillation, said method comprising
a) determining the amount of GDF-15 in a sample of said subject; and

b) comparing the amount of GDF-15 determined in step a) with a suitable reference amount whereby heart failure is to be diagnosed.

In a preferred embodiment of this method, it further (i.e., in addition to the determination of GDF-15) comprises the steps of determining the amount of a natriuretic peptide in said sample of the subject and comparing the amount of the natriuretic peptide to a reference. Said further steps may be carried out simultaneously or prior or subsequently to the determination of GDF-15 according to the method of the present invention.

It has revealed, however, that elevated GDF-15 levels can also be caused by diseases other than cardiac disorders, namely pulmonary-, heart- or kidney malfunctions. GDF-15 is, hence, not specific for cardiac disorders, which may result in false positive results for patients not suffering from cardiac disorders. Patients often are in need of a supportive therapy for the cardiac disorder, in particular myocardial dysfunction, heart failure and/or acute coronary syndrome (ACS), without loosing time in further diagnoses. Accordingly, there is a need for diagnostic measures which allow a reliable and fast diagnosis of cardiac disorders, in particular myocardial dysfunction, heart failure and/or ACS in patients showing elevated GDF-15 levels, in order to allow for an efficient medical treatment regimen.

The technical problem underlying the present invention can be seen as the provision of means and methods for complying with the aforementioned needs. The technical problem is solved by the embodiments characterized in the claims and herein below.

Accordingly, the present invention relates to a method of diagnosing a cardiac disorder in a subject exhibiting atrial fibrillation, said method comprising
a) determining the amount of a natriuretic peptide in a sample of said subject;
b) determining the amount of GDF-15 in a sample of said subject;
c) calculating the ratio (natriuretic peptide/GDF-15);
d) diagnosing if the subject is suffering from a cardiac disorder, based of the ratio calculated in step c).

The method of the present invention, preferably, is an in vitro method. Moreover, it may comprise steps in addition to those explicitly mentioned above. For example, further steps may relate to sample pre-treatments or evaluation of the results obtained by the method. The method of the present invention may be also used for monitoring, confirmation, and subclassification of a subject. The method may be carried out manually or assisted by automation. Preferably, step (a) and/or (b) and/or (c) and/or (d) may in total or in part be assisted by automation, e.g., by a suitable robotic and sensory equipment for the determination in steps (a) and/or (b) or a computer-implemented comparison in step (c).

The term “diagnosing” as used herein means assessing as to whether a subject having an elevated level of a natriuretic peptide and/or GDF-15 suffers from a cardiac disorder, in particular heart failure, or not. As will be understood by those skilled in the art, such an assessment is usually not intended to be correct for all (i.e., 100%) of the subjects to be identified. The term, however, requires that a statistically significant portion of subjects can be identified (e.g., a cohort in a cohort study). Whether a portion is statistically significant can be determined without further ado by the person skilled in the art using various well known statistic evaluation tools, e.g., determination of confidence intervals, p-value determination, Student's t-test, Mann-Whitney test etc. Details are found in Dowdy and Wearden, Statistics for Research, John Wiley & Sons, New York 1983. Preferred confidence intervals are at least 90%, at least 95%, at least 97%, at least 98% or at least 99%. The p-values are, preferably, 0.1, 0.05, 0.01, 0.005, or 0.0001. More preferably, at least 60%, at least 70%, at least 80% or at least 90% of the subjects of a population can be properly identified by the method, of the present invention.

Diagnosing according to the present invention includes monitoring, confirmation, subclassification and prediction of the relevant disease, symptoms or risks therefor. Monitoring relates to keeping track of an already diagnosed disease. Confirmation relates to the strengthening or substantiating a diagnosis already performed using other indicators or markers. Subclassification relates to further defining a diagnosis according to different subclasses of the diagnosed disease, e.g., defining according to mild and severe forms of the disease.

The term “subject” as used herein relates to animals, preferably mammals, and, more preferably, humans. Preferably, the subject referred to in accordance with the aforementioned method suffers from a myocardial disorder and/or heart failure or exhibits the symptoms or clinical parameters, such as an increased NT-proBNP level, accompanied therewith, i.e., being at least suspect to suffer from a myocardial disorder and/or heart failure.

GDF-15 is a member of the TGF-beta family. GDF-15 is elevated in subjects having cardiac disorders. However, GDF-15 has been found to not be specific for cardiac disorders, but is also elevated in malfunctions of the kidney, liver and lung, see, e.g., Proc. Natl. Acad. Sci. USA, Vol 94, pp 11514-11519, 1997. Likewise, GDF-15 has been found to be elevated in certain tumours. Typical examples include ovarian cancer and all forms of epithelial cancers, in particular colorectal cancer, prostate cancer, breast cancer and gastric carcinoma.

Therefore, it is not possible to diagnose, from the GDF-15 level alone, if a subject with an elevated GDF-15 level is suffering from a cardiac disorder, or if it is suffering from another disease, possibly in connection with a cardiac disorder.

In order to establish a diagnosis if the elevated GDF-15 level is related to a cardiac disorder, the present invention teaches to measure the level of a natriuretic peptide, further to GDF-15. From the ratio natriuretic peptide/GDF-15 which is calculated from the values obtained, it is clear whether the subject is suffering from a cardiac disorder, or is suffering from a different disease.

According to the present invention, a high ratio (natriuretic peptide/GDF-15) is indicative for a myocardial dysfunction. The person skilled in the art is aware that the values indicating a myocardial dysfunction or not may vary, according to the natriuretic peptide the level of which is determined.

The following values are values which have been established for NT-proBNP. Here, preferably, a ratio of ≧0.10 (higher than or equal to 0.10) is indicative for a myocardial dysfunction. A ratio of ≧0.12 (higher than or equal to 0.12) indicates a high probability for the occurrence of a myocardial dysfunction. A ratio of ≧0.15 (higher than or equal to 0.15) indicates a very high probability for the occurrence of a myocardial dysfunction A ratio (natriuretic peptide/GDF-15) of <0.10 (below 0.10) indicates that the GDF-15 value is not associated with a myocardial dysfunction.

It is known that the for the same pathophysiological conditions, the concentrations of BNP are found the be 6 to 8 times lower than those for NT-proBNP. Accordingly, preferably, a ratio of ≧0.015 (higher than or equal to 0.015) is indicative for a myocardial dysfunction. A ratio of ≧0.017 (higher than or equal to 0.017) indicates a high probability for the occurrence of a myocardial dysfunction. A ratio of ≧0.021 (higher than or equal to 0.021) indicates a very high probability for the occurrence of a myocardial dysfunction A ratio (natriuretic peptide/GDF-15) of <0.015 (below 0.015) indicates that the GDF-15 value is not associated with a myocardial dysfunction.

These values may apply for the other natriuretic peptides as well. They may also be different. In the knowledge of the present invention, however, the person skilled in the art knows to adapt the values given above to the other natriuretic peptides, by applying values published in the prior art, or in a routine measurement replacing NT-proBNP for another natriuretic peptide.

The European patent application 07108852.0 of the applicant, also mentioned beforehand, discloses a a method of diagnosing heart failure in a subject exhibiting atrial fibrillation, comprising determining the amount of GDF-15 in a sample of said subject; and comparing the amount of GDF-15 with a suitable reference amount whereby heart failure is to be diagnosed, wherein, in a preferred embodiment of the method of the invention, the method further (i.e., in addition to the determination of GDF-15) comprises the steps of determining the amount of a natriuretic peptide in said sample of the subject and comparing the amount of the natriuretic peptide to a reference. Said further steps may be carried out simultaneously or prior or subsequently to the determination of GDF-15 according to the method of the present invention. More preferably, the natriuretic peptide is initially determined and heart failure will be confirmed as described beforehand by a subsequent GDF-15 determination. However, this patent application does not describe calculating the ratio of the natriuretic peptide to GDF-15 and diagnosing, on this basis, if the subject is suffering from a cardiac disorder.

The method according to the present, invention comprises determining the amount of GDF-15 in a sample of said subject, and determining the amount of a natriuretic peptide in a sample of the subject. These steps may be carried out simultaneously, or prior or subsequently.

The term “natriuretic peptide” comprises atrial natriuretic peptide (ANP)-type and brain natriuretic peptide (BNP)-type peptides and variants thereof having the same predictive potential. Natriuretic peptides according to the present invention comprise ANP-type and BNP-type peptides and variants thereof (see, e.g., Bonow, 1996, Circulation 93: 1946-1950). ANP-type peptides comprise pre-proANP, proANP, NT-proANP, and ANP. BNP-type peptides comprise pre-proBNP, proBNP, NT-proBNP, and BNP. The pre-pro peptide (134 amino acids in the case of pre-proBNP) comprises a short signal peptide, which is enzymatically cleaved off to release the pro peptide (108 amino acids in the case of proBNP). The pro peptide is further cleaved into an N-terminal pro peptide (NT-pro peptide, 76 amino acids in case of NT-proBNP) and the active hormone (32 amino acids in the case of BNP, 28 amino acids in the case of ANP). Preferred natriuretic peptides according to the present invention are NT-proANP, ANP, NT-proBNP, BNP, and variants thereof. ANP and BNP are the active hormones and have a shorter half-life than their respective inactive counterparts, NT-proANP and NT-proBNP. BNP is metabolised in the blood, whereas NT-proBNP circulates in the blood as an intact molecule and as such is eliminated renally. The in-vivo half-life of NTproBNP is 120 min longer than that of BNP, which is 20 min (Smith 2000, J Endocrinol. 167: 239-46). Preanalytics are more robust with NT-proBNP allowing easy transportation of the sample to a central laboratory (Mueller 2004, Clin Chem Lab Med 42: 942-4). Blood samples can be stored at room temperature for several days or may be mailed or shipped without recovery loss. In contrast, storage of BNP for 48 hours at room temperature or at 4° Celsius leads to a concentration loss of at least 20% (Mueller loc. cit.; Wu 2004, Clin Chem 50: 867-73). Therefore, depending on the time-course or properties of interest, either measurement of the active or the inactive forms of the natriuretic peptide can be advantageous. More preferred natriuretic peptides according to the present invention are BNP and NT-proBNP or variants thereof. The most preferred natriuretic peptides according to the present invention are NT-proBNP or variants thereof. As briefly discussed above, the human NT-proBNP, as referred to in accordance with the present invention, is a polypeptide comprising, preferably, 76 amino acids in length corresponding to the N-terminal portion of the human NT-proBNP molecule. The structure of the human BNP and NT-proBNP has been described already in detail in the prior art, e.g., WO 02/089657, WO 02/083913 or Bonow loc. cit. Preferably, human NT-proBNP as used herein is human NT-proBNP as disclosed in EP 0 648 228 B1. These prior art documents are herewith incorporated by reference with respect to the specific sequences of NT-proBNP and variants thereof disclosed therein. The NT-proBNP referred to in accordance with the present invention further encompasses allelic and other variants of said specific sequence for human NT-proBNP discussed above. Specifically, envisaged are variant polypeptides which are on the amino acid level at least 60% identical, more preferably at least 70%, at least 80%, at least 90%, at least 95%, at least 98% or at least 99% identical, to human NT-proBNP. Substantially similar and also envisaged are proteolytic degradation products which are still recognized by the diagnostic means or by ligands directed against the respective full-length peptide. Also encompassed are variant polypeptides having amino acid deletions, substitutions, and/or additions compared to the amino acid sequence of human NT-proBNP as long as the said polypeptides have NT-proBNP properties. NT-proBNP properties as referred to herein are immunological and/or biological properties. Preferably, the NT-proBNP variants have immunological properties (i.e., epitope composition) comparable to those of NT-proBNP. Thus, the variants shall be recognizable by the aforementioned means or ligands used for determination of the amount of the natriuretic peptides. Biological and/or immunological NT-proBNP properties can be detected by the assay described in Karl et al. (Karl 1999, Scand J Clin Invest 230:177-181), Yeo et al. (Yeo 2003, Clinica Chimica Acta 338:107-115). Variants also include posttranslationally modified peptides such as glycosylated peptides. Further, a variant in accordance with the present invention is also a peptide or polypeptide which has been modified after collection of the sample, for example by covalent or non-covalent attachment of a label, particularly a radioactive or fluorescent label, to the peptide.

As discussed above already, a preferred reference amount serving as a threshold may be derived from the ULN. The ULN for a given population of subjects can be determined as specified elsewhere in this description. A preferred threshold (i.e., reference amount) for a natriuretic peptide and, in particular for NT-proBNP, is at least one times, more preferably two to four times the ULN. Preferably, the ULN for NT-proBNP referred to in this context is 125 pg/ml. ULNs for the other natriuretic peptides are known in the art and are, preferably, 40 pg/ml for ANP, 50 pg/ml for BNP and 800 pg/ml for NT-proANP. An amount of a natriuretic peptide larger than the reference amount is, more preferably, additionally indicative for a subject suffering from heart failure.

The present invention in relates to cardiac disorders, preferably from the group myocardial dysfunction and heart failure.

The term “myocardial dysfunction” as used herein is a general term and relates to several pathological states of the myocard. A myocardial dysfunction may be a temporary pathological state (caused by, e.g., ischemia, toxic substances, alcohol, . . . ). Myocardial dysfunction may disappear after removing the underlying cause. In the context of the present invention, the myocardial dysfunction can be a symptomless myocardial dysfunction. A myocardial dysfunction, in particular a symptomless myocardial dysfunction, may also develop into heart failure. A myocardial dysfunction may also be a severe chronic heart failure. In general, a myocardial dysfunction is an impaired systolic and/or diastolic function of the heart, and a myocardial dysfunction may occur with or without heart failure.

The term “heart failure” as used herein relates to an impaired systolic and/or diastolic function of the heart. Preferably, heart failure referred to herein is also chronic heart failure. Heart failure can be classified into a functional classification system according to the New York Heart Association (NYHA). Patients of NYHA Class I have no obvious symptoms of cardiovascular disease but already have objective evidence of functional impairment. Physical activity is not limited, and ordinary physical activity does not cause undue fatigue, palpitation, or dyspnea (shortness of breath). Patients of NYHA class II have slight limitation of physical activity. They are comfortable at rest, but ordinary physical activity results in fatigue, palpitation, or dyspnea. Patients of NYHA class III show a marked limitation of physical activity. They are comfortable at rest, but less than ordinary activity causes fatigue, palpitation, or dyspnea. Patients of NYHA class IV are unable to carry out any physical activity without discomfort. They show symptoms of cardiac insufficiency at rest. Heart failure, i.e., an impaired systolic and/or diastolic function of the heart, can be determined also by, for example, echocardiography, angiography, szintigraphy, or magnetic resonance imaging. This functional impairment can be accompanied by symptoms of heart failure as outlined above (NYHA class II-IV), although some patients may present without significant symptoms (NYHA I). Moreover, heart failure is also apparent by a reduced left ventricular ejection fraction (LVEF). More preferably, heart failure as used herein is accompanied by a left ventricular ejection fraction (LVEF) of less than 60%, of 40% to 60% or of less than 40%.

The term “acute coronary syndrome (ACS) is known to the person skilled in the art. ACS patients can show unstable angina pectoris (UAP) or these individuals have already suffered from a myocardial infarction (MI). MI can be an ST-elevated MI or a non-ST-elevated MI. The occurring of an MI can be followed by a left ventricular dysfunction (LVD). Finally, LVD patients undergo congestive heart failure (CHF) with a considerable mortality rate.

The term “growth-differentiation factor-15” or “GDF-15” relates to a polypeptide being a member of the transforming growth factor (TGF)-β cytokine superfamily. The terms polypeptide, peptide and protein are used interchangeable throughout this specification. GDF-15 was originally cloned as macrophage-inhibitory cytokine-1 and later also identified as placental transforming growth factor-β, placental bone morphogenetic protein, non-steroidal anti-inflammatory drug-activated gene-I, and prostate-derived factor (Bootcov loc cit; Hromas, 1997 Biochim Biophys Acta 1354:40-44; Lawton 1997, Gene 203:17-26; Yokoyama-Kobayashi 1997, J Biochem (Tokyo), 122:622-626; Paralkar 1998, J Biol Chem 273:13760-13767). Similar to other TGF-β-related cytokines, GDF-15 is synthesized as an inactive precursor protein, which undergoes disulfide-linked homodimerization. Upon proteolytic cleavage of the N-terminal pro-peptide, GDF-15 is secreted as a ˜28 kDa dimeric protein (Bauskin 2000, Embo J 19:2212-2220). Amino acid sequences for GDF-15 are disclosed in WO99/06445, WO00/70051, WO2005/113585, Bottner 1999, Gene 237: 105-111, Bootcov loc. cit, Tan loc. cit., Baek 2001, Mol Pharmacol 59: 901-908, Hromas loc cit, Paralkar loc cit, Morrish 1996, Placenta 17:431-441 or Yokoyama-Kobayashi loc cit. GDF-15 as used herein encompasses also variants of the aforementioned specific GDF-15 polypeptides. Such variants have at least the same essential biological and immunological properties as the specific GDF-15 polypeptides. In particular, they share the same essential biological and immunological properties if they are detectable by the same specific assays referred to in this specification, e.g., by ELISA assays using polyclonal or monoclonal antibodies specifically recognizing the said GDF-15 polypeptides. A preferred assay is described in the accompanying Examples. Moreover, it is to be understood that a variant as referred to in accordance with the present invention shall have an amino acid sequence which differs due to at least one amino acid substitution, deletion and/or addition wherein the amino acid sequence of the variant is still, preferably, at least 50%, 60%, 70%, 80%, 85%, 90%, 92%, 95%, 97%, 98%, or 99% identical with the amino sequence of the specific GDF-15 polypeptides. The degree of identity between two amino acid sequences can be determined by algorithms well known in the art. Preferably, the degree of identity is to be determined by comparing two optimally aligned sequences over a comparison window, where the fragment of amino acid sequence in the comparison window may comprise additions or deletions (e.g., gaps or overhangs) as compared to the reference sequence (which does not comprise additions or deletions) for optimal alignment. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad Sci. (USA) 85: 2444 (1988), by computerized implementations of these algorithms (GAP, BESTFIT, BLAST, PASTA, and TFASTA in the Wisconsin Genetics Software Package, Genetics Computer Group (GCG), 575 Science Dr., Madison, Wis.), or by visual inspection. Given that two sequences have been identified for comparison, GAP and BESTFIT are preferably employed to determine their optimal alignment and, thus, the degree of identity. Preferably, the default values of 5.00 for gap weight and 0.30 for gap weight length are used. Variants referred to above may be allelic variants or any other species specific homologs, paralogs, or orthologs. Moreover, the variants referred to herein include fragments of the specific GDF-15 polypeptides or the aforementioned types of variants as long as these fragments have the essential immunological and biological properties as referred to above. Such fragments may be, e.g., degradation products of the GDF-15 polypeptides. Further included are variants which differ due to posttranslational modifications such as phosphorylation or myristylation.

The term “sample” refers to a sample of a body fluid, to a sample of separated cells or to a sample from a tissue or an organ. Samples of body fluids can be obtained by well known techniques and include, preferably, samples of blood, plasma, serum, or urine, more preferably, samples of blood, plasma or serum. Tissue or organ samples may be obtained from any tissue or organ by, e.g., biopsy. Separated cells may be obtained from the body fluids or the tissues or organs by separating techniques such as centrifugation or cell sorting. Preferably, cell-, tissue- or organ samples are obtained from those cells, tissues or organs which express or produce the peptides referred to herein.

Determining the amount of the peptides or polypeptides referred to in this specification relates to measuring the amount or concentration, preferably semi-quantitatively or quantitatively. Measuring can be done directly or indirectly. Direct measuring relates to measuring the amount or concentration of the peptide or polypeptide based on a signal which is obtained from the peptide or polypeptide itself and the intensity of which directly correlates with the number of molecules of the peptide present in the sample. Such a signal—sometimes referred to herein as intensity signal—may be obtained, e.g., by measuring an intensity value of a specific physical or chemical property of the peptide or polypeptide. Indirect measuring includes measuring of a signal obtained from a secondary component (i.e., a component not being the peptide or polypeptide itself) or a biological read out system, e.g., measurable cellular responses, ligands, labels, or enzymatic reaction products.

In accordance with the present invention, determining the amount of a peptide or polypeptide can be achieved by all known means for determining the amount of a peptide in a sample. Said means comprise immunoassay devices and methods which may utilize labeled molecules in various sandwich, competition, or other assay formats. Said assays will develop a signal which is indicative for the presence or absence of the peptide or polypeptide. Moreover, the signal strength can, preferably, be correlated directly or indirectly (e.g., reverse-proportional) to the amount of polypeptide present in a sample. Further suitable methods comprise measuring a physical or chemical property specific for the peptide or polypeptide such as its precise molecular mass or NMR spectrum. Said methods comprise, preferably, biosensors, optical devices coupled to immunoassays, biochips, analytical devices such as mass-spectrometers, NMR-analyzers, or chromatography devices. Further, methods include micro-plate ELISA-based methods, fully-automated or robotic immunoassays (available for example on ELECSYS analyzers, Roche Diagnostics GmbH), CBA (an enzymatic cobalt binding assay, available, for example, on Roche-Hitachi analyzers), and latex agglutination assays (available, for example, on Roche-Hitachi analyzers).

Preferably, determining the amount of a peptide or polypeptide comprises the steps of (a) contacting a cell capable of eliciting a cellular response the intensity of which is indicative of the amount of the peptide or polypeptide with the said peptide or polypeptide for an adequate period of time, (b) measuring the cellular response. For measuring cellular responses, the sample or processed sample is, preferably, added to a cell culture and an internal or external cellular response is measured. The cellular response may include the measurable expression of a reporter gene or the secretion of a substance, e.g., a peptide, polypeptide, or a small molecule. The expression or substance shall generate an intensity signal which correlates to the amount of the peptide or polypeptide.

Also preferably, determining the amount of a peptide or polypeptide comprises the step of measuring a specific intensity signal obtainable from the peptide or polypeptide in the sample. As described above, such a signal may be the signal intensity observed at an m/z variable specific for the peptide or polypeptide observed in mass spectra or a NMR spectrum specific for the peptide or polypeptide.

Determining the amount of a peptide or polypeptide may, preferably, comprises the steps of (a) contacting the peptide with a specific ligand, (b) (optionally) removing non-bound ligand, (c) measuring the amount of bound ligand. The bound ligand will generate an intensity signal. Binding according to the present invention includes both covalent and non-covalent binding. A ligand according to the present invention can be any compound, e.g., a peptide, polypeptide, nucleic acid, or small molecule, binding to the peptide or polypeptide described herein. Preferred ligands include antibodies, nucleic acids, peptides or polypeptides such as receptors or binding partners for the peptide or polypeptide and fragments thereof comprising the binding domains for the peptides, and aptamers, e.g., nucleic acid or peptide aptamers. Methods to prepare such ligands are well-known in the art. For example, identification and production of suitable antibodies or aptamers is also offered by commercial suppliers. The person skilled in the art is familiar with methods to develop derivatives of such ligands with higher affinity or specificity. For example, random mutations can be introduced into the nucleic acids, peptides or polypeptides. These derivatives can then be tested for binding according to screening procedures known in the art, e.g., phage display. Antibodies as referred to herein include both polyclonal and monoclonal antibodies, as well as fragments thereof, such as Fv, Fab and F(ab)₂ fragments that are capable of binding antigen or hapten. The present invention also includes single chain antibodies and humanized hybrid antibodies wherein amino acid sequences of a non-human donor antibody exhibiting a desired antigen-specificity are combined with sequences of a human acceptor antibody. The donor sequences will usually include at least the antigen-binding amino acid residues of the donor but may comprise other structurally and/or functionally relevant amino acid residues of the donor antibody as well. Such hybrids can be prepared by several methods well known in the art. Preferably, the ligand or agent binds specifically to the peptide or polypeptide. Specific binding according to the present invention means that the ligand or agent should not bind substantially to (“cross-react” with) another peptide, polypeptide or substance present in the sample to be analyzed. Preferably, the specifically bound peptide or polypeptide should be bound with at least 3 times higher, more preferably at least 10 times higher and even more preferably at least 50 times higher affinity than any other relevant peptide or polypeptide. Non-specific binding may be tolerable, if it can still be distinguished and measured unequivocally, e.g., according to its size on a Western Blot, or by its relatively higher abundance in the sample. Binding of the ligand can be measured by any method known in the art. Preferably, said method is semi-quantitative or quantitative. Suitable methods are described in the following.

First, binding of a ligand may be measured directly, e.g., by NMR or surface plasmon resonance.

Second, if the ligand also serves as a substrate of an enzymatic activity of the peptide or polypeptide of interest, an enzymatic reaction product may be measured (e.g., the amount of a protease can be measured by measuring the amount of cleaved substrate, e.g., on a Western Blot). Alternatively, the ligand may exhibit enzymatic properties itself and the “ligand/peptide or polypeptide” complex or the ligand which was bound by the peptide or polypeptide, respectively, may be contacted with a suitable substrate allowing detection by the generation of an intensity signal. For measurement of enzymatic reaction products, preferably the amount of substrate is saturating. The substrate may also be labeled with a detectable table prior to the reaction. Preferably, the sample is contacted with the substrate for an adequate period of time. An adequate period of time refers to the time necessary for an detectable, preferably measurable, amount of product to be produced. Instead of measuring the amount of product, the time necessary for appearance of a given (e.g., detectable) amount of product can be measured.

Third, the ligand may be coupled covalently or non-covalently to a label allowing detection and measurement of the ligand. Labeling may be done by direct or indirect methods. Direct labeling involves coupling of the label directly (covalently or non-covalently) to the ligand. Indirect labeling involves binding (covalently or non-covalently) of a secondary ligand to the first ligand. The secondary ligand should specifically bind to the first ligand. Said secondary ligand may be coupled with a suitable label and/or be the target (receptor) of tertiary ligand binding to the secondary ligand. The use of secondary, tertiary or even higher order ligands is often used to increase the signal. Suitable secondary and higher order ligands may include antibodies, secondary antibodies, and the well-known streptavidin-biotin system (Vector Laboratories, Inc.). The ligand or substrate may also be “tagged” with one or more tags as known in the art. Such tags may then be targets for higher order ligands. Suitable tags include biotin, digoxigenin, His-tag, glutathione-S-transferase, FLAG, GFP, myc-tag, influenza A virus haemagglutinin (HA), maltose binding protein, and the like. In the case of a peptide or polypeptide, the tag is preferably at the N-terminus and/or C-terminus. Suitable labels are any labels detectable by an appropriate detection method. Typical labels include gold particles, latex beads, acridan ester, luminol, ruthenium, enzymatically active labels, radioactive labels, magnetic labels (“e.g., magnetic beads”, including paramagnetic and superparamagnetic labels), and fluorescent labels. Enzymatically active labels include, e.g., horseradish peroxidase, alkaline phosphatase, beta-Galactosidase, Luciferase, and derivatives thereof. Suitable substrates for detection include di-amino-benzidine (DAB), 3,3′-5,5′-tetramethylbenzidine, NBT-BCIP (4-nitro blue tetrazolium chloride and 5-bromo-4-chloro-3-indolyl-phosphate, available as ready-made stock solution from Roche Diagnostics), CDP-Star (Amersham Biosciences), ECF (Amersham Biosciences). A suitable enzyme-substrate combination may result in a colored reaction product, fluorescence or chemiluminescence, which can be measured according to methods known in the art (e.g., using a light-sensitive film or a suitable camera system). As for measuring the enzymatic reaction, the criteria given above apply analogously. Typical fluorescent labels include fluorescent proteins (such as GFP and its derivatives), Cy3, Cy5, Texas Red, Fluorescein, and the Alexa dyes (e.g., Alexa 568). Further fluorescent labels are available, e.g., from Molecular Probes (Oregon). Also the use of quantum dots as fluorescent labels is contemplated. Typical radioactive labels include ³⁵S, ¹²⁵I, ³²P, ³³P and the like. A radioactive label can be detected by any method known and appropriate, e.g., a light-sensitive film or a phosphor imager. Suitable measurement methods according the present invention also include precipitation (particularly immunoprecipitation), electrochemiluminescence (electro-generated chemiluminescence), RIA (radioimmunoassay), ELISA (enzyme-linked immunosorbent assay), sandwich enzyme immune tests, electrochemiluminescence sandwich immunoassays (ECLIA), dissociation-enhanced lanthanide fluoroimmuno assay (DELFIA), scintillation proximity assay (SPA), turbidimetry, nephelometry, latex-enhanced turbidimetry or nephelometry, or solid phase immune tests. Further methods known in the art (such as gel electrophoresis, 2D gel electrophoresis, SDS polyacrylamide gel electrophoresis (SDS-PAGE), Western Blotting, and mass spectrometry), can be used alone or in combination with labeling or other detection methods as described above.

The amount of a peptide or polypeptide may be, also preferably, determined as follows: (a) contacting a solid support comprising a ligand for the peptide or polypeptide as specified above with a sample comprising the peptide or polypeptide and (b) measuring the amount peptide or polypeptide which is bound to the support. The ligand, preferably chosen from the group consisting of nucleic acids, peptides, polypeptides, antibodies and aptamers, is preferably present on a solid support in immobilized form. Materials for manufacturing solid supports are well known in the art and include, inter alia, commercially available column materials, polystyrene beads, latex beads, magnetic beads, colloid metal particles, glass and/or silicon chips and surfaces, nitrocellulose strips, membranes, sheets, duracytes, wells and walls of reaction trays, plastic tubes etc. The ligand or agent may be bound to many different carriers. Examples of well-known carriers include glass, polystyrene, polyvinyl chloride, polypropylene, polyethylene, polycarbonate, dextran, nylon, amyloses, natural and modified celluloses, polyacrylamides, agaroses, and magnetite. The nature of the carrier can be either soluble or insoluble for the purposes of the invention. Suitable methods for fixing/immobilizing said ligand are well known and include, but are not limited to ionic, hydrophobic, covalent interactions and the like. It is also contemplated to use “suspension arrays” as arrays according to the present invention (Nolan 2002, Trends Biotechnol. 20(1):9-12). In such suspension arrays, the carrier, e.g., a microbead or microsphere, is present in suspension. The array consists of different microbeads or microspheres, possibly labeled, carrying different ligands. Methods of producing such arrays, for example based on solid-phase chemistry and photo-labile protective groups, are generally known (U.S. Pat. No. 5,744,305).

The term “amount” as used herein encompasses the absolute amount of a polypeptide or peptide, the relative amount or concentration of the said polypeptide or peptide as well as any value or parameter which correlates thereto or can be derived therefrom. Such values or parameters comprise intensity signal values from all specific physical or chemical properties obtained from the said peptides by direct measurements, e.g., intensity values in mass spectra or NMR spectra. Moreover, encompassed are all values or parameters which are obtained by indirect measurements specified elsewhere in this description, e.g., response levels determined from biological read out systems in response to the peptides or intensity signals obtained from specifically bound ligands. It is to be understood that values correlating to the aforementioned amounts or parameters can also be obtained by all standard mathematical operations.

Based on the method of the present invention, myocardial dysfunction and hidden heart failure (i.e., heart failure which remains unrecognized because the currently applied diagnostic standards are disregarded) can be treated more efficiently. The method of the present invention, advantageously, allows for a reliable, fast and less cost intensive diagnosis and can be implemented even in portable assays, such as test strips. Therefore, the method is particularly well suited for diagnosing emergency patients. Thanks to the findings of the present invention, a suitable therapy for a subject can be timely and reliably selected, e.g., a therapy for heart failure. Severe side effects caused by the late and/or wrong treatment of patients can be avoided.

The present invention, furthermore, relates to a device for diagnosing a cardiac disorder in a subject exhibiting atrial fibrillation comprising
a) means for determining the amount of a natriuretic peptide in a sample of said subject;
b) means for determining the amount of GDF-15 in a sample of said subject;
c) optionally means for calculating the ratio (natriuretic peptide/GDF-15);
d) optionally means for diagnosing if the subject is suffering from a cardiac disorder, based of the ratio calculated in step c).

The term “device” as used herein relates to a system of means comprising at least the aforementioned means operatively linked to each other as to allow the diagnosis. Preferred means for determining the amount of GDF-15 and means for determining the amount of a natriuretic peptide, and means for calculating and diagnosing if the subject is suffering from a cardiovascular disorder are disclosed above in connection with the method of the invention. How to link the means in an operating manner will depend on the type of means included into the device. For example, where means for automatically determining the amount of the peptides are applied, the data obtained by said automatically operating means can be processed by, e.g., a computer program in order to obtain the desired results. Preferably, the means are comprised by a single device in such a case. Said device may accordingly include an analyzing unit for the measurement of the amount of the peptides or polypeptides in an applied sample and a computer unit for processing the resulting data for the evaluation. Alternatively, where means such as test strips are used for determining the amount of the peptides or polypeptides, the means for comparison may comprise control strips or tables allocating the determined amount to a reference amount. The test strips are, preferably, coupled to a ligand which specifically binds to the peptides or polypeptides referred to herein. The strip or device, preferably, comprises means for detection of the binding of said peptides or polypeptides to the said ligand. Preferred means for detection are disclosed in connection with embodiments relating to the method of the invention above. In such a case, the means are operatively linked in that the user of the system brings together the result of the determination of the amount and the diagnostic or prognostic value thereof due to the instructions and interpretations given in a manual. The means may appear as separate devices in such an embodiment and are, preferably, packaged together as a kit. The person skilled in the art will realize how to link the means without further ado. Preferred devices are those which can be applied without the particular knowledge of a specialized clinician, e.g., test strips or electronic devices which merely require loading with a sample. The results may be given as output of raw data which need interpretation by the clinician. Preferably, the output of the device is, however, processed, i.e., evaluated, raw data the interpretation of which does not require a clinician. Further preferred devices comprise the analyzing units/devices (e.g., biosensors, arrays, solid supports coupled to ligands specifically recognizing the natriuretic peptide, Plasmon surface resonance devices, NMR spectrometers, mass-spectrometers etc.) or evaluation units/devices referred to above in accordance with the method of the invention.

Finally, the present invention relates to a kit adapted for carrying out the method of the present invention wherein said kit comprises instructions for carrying out the said method and
a) means for determining the amount of a natriuretic peptide in a sample of said subject;
b) means for determining the amount of GDF-15 in a sample of said subject;
c) optionally means for calculating the ratio (natriuretic peptide/GDF-15);
d) optionally means for diagnosing if the subject is suffering from a cardiac disorder, based of the ratio calculated in step c).

The term “kit” as used herein refers to a collection of the aforementioned means, preferably, provided in separately or within a single container. The container, also preferably, comprises instructions for carrying out the method of the present invention. Accordingly, a kit adopted for carrying out the method of the present invention comprises all components required for practicing said method in an ready-to-use manner, e.g., in a premixed form with adjusted concentrations of the components used for determination and/or comparison.

All references cited in this specification are herewith incorporated by reference with respect to their entire disclosure content and the disclosure content specifically mentioned in this specification.

The following Examples shall merely illustrate the invention. They shall not be construed, whatsoever, to limit the scope of the invention.

### EXAMPLE 1

### Determination of GDF-15 and NT-proBNP in Serum and Plasma Samples

To determine the concentration of GDF-15 in serum and plasma samples, an immunoradiometric assay (IRMA) using a polyclonal, GDF-15 affinity chromatography-purified, goat anti-human GDF-15 IgG antibody from R&D Systems (AF957) was developed. Maxisorp Startubes (Nunc) were coated overnight at 4° C. with 0.5 μg anti-GDF-15 IgG in 0.1 M Na-carbonate buffer (pH 9.0), and then washed twice with phosphate-buffered saline with 0.1% Tween 20. Serum or plasma samples (100 μl) were diluted 1:1 with assay buffer (30 g/l BSA, 10 g/l bovine IgG, 1% goat serum, 0.1% Na-azide, 1 M NaCl, 40 mM Na phosphate buffer, pH 7.4), added to the tubes, and incubated for 16 hours at 4° C. After two washing steps, 10 ng of [125I]-iodinated anti-GDF-15 IgG (specific activity 0.74 MBq/μg) were diluted in 200 μl assay buffer, added to each tube, and incubated for 4 hours at room temperature. After three final washing steps, bound radioactivity was quantified in a gamma counter (LKB Wallac 1261). In each experiment, a standard curve was generated with recombinant human GDF-15 from R&D Systems (957-GD/CF). The results with new batches of recombinant GDF-15 protein were tested in standard plasma samples and any deviation above 10% was corrected by introducing an adjustment factor for this assay. GDF-15 measurements in serum and plasma samples from the same patient yielded virtually identical results after correction for eventual dilution factors. The detection limit of the assay was 20 pg/ml. The intraassay coefficient of variation determined for mean GDF-15 levels of 744, 1518, and 8618 pg/ml was 5.6, 5.9, and 6.5%, respectively. The inter-assay coefficient of variation determined for mean GDF-15 levels of 832, 4739, and 9230 pg/ml was 8.6, 5.7, and 4.4%, respectively.

NT-proBNP levels were determined with an immunoassay on an ELECSYS 2010 with a detection limit of 20 pg/ml.

### EXAMPLE 2

In a cohort of patients of which 50 had cancer, 76 had liver fibrosis, 120 apparently healthy blood donors, and the rest having various cardiac disorders, GDF-15 and NT-proBNP were determined. An NT-proBNP value of below 150 pg/ml was taken as a hint for the non-occurrence of a myocardial dysfunction (heart failure).

For every patient, the ratio NT-proBNP/GDF-15 was calculated. In persons having a proven myocardial dysfunction (NT-proBNP value of higher 150 pg/ml), the ratio NT-proBNP/GDF-15 was higher than 0.1. In persons having an elevated level of GDF-15 but having apparently no myocardial dysfunction, the ratio NT-proBNP/GDF-15 was below 0.1.

The plasma levels of GDF-15 and NT-proBNP were determined as described in Example 1 above.

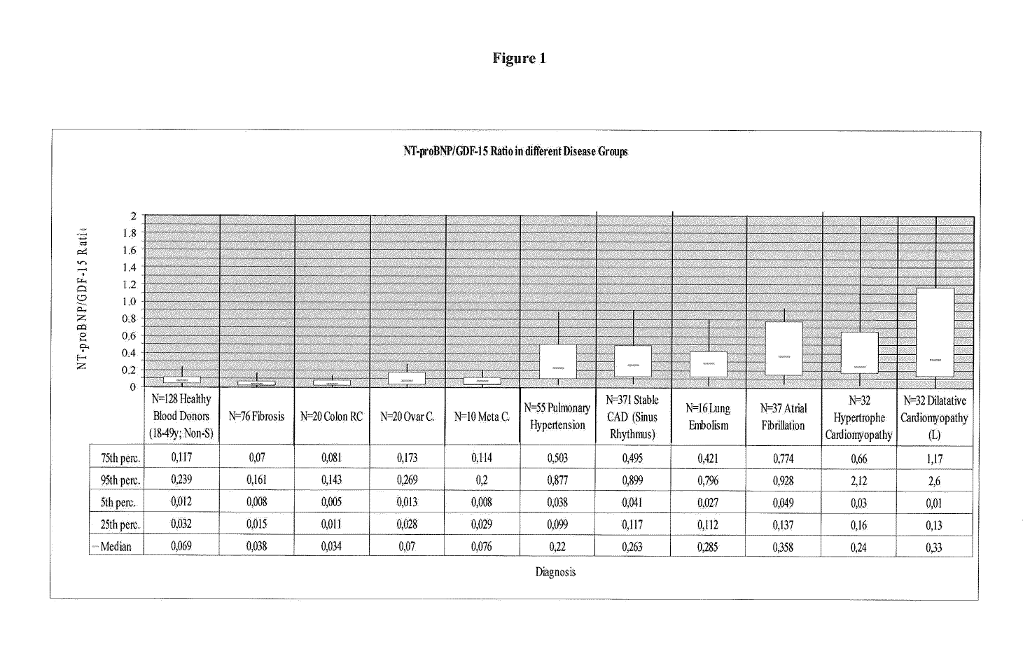

The results of the study are shown in the following table and FIG. 1:

| | Median | | Median |
|---|---|---|---|
| | NT- | Median | NT-proBNP/ |
| | proBNP | GDF-15 | GDF-15 |
| Disease Group | [pg/ml] | [pg/ml] | Ratio |
| Pulmonary Embolism (N = 16) | 3599 | 2215 | 0.285 |
| Atrial Fibrillation (N = 26) | 1164 | 866 | 0.358 |
| Pulmonary Hypertension (N = 55) | 690 | 1580 | 0.220 |
| Stable CAD (Sinus Rhythm) (N = 223) | 239 | 679 | 0.263 |
| Cardiomyopathy, dilatative (N = 32) | 359 | 1241 | 0.330 |
| Cardiomyopathy, hypertrophic (N = 29) | 282 | 1031 | 0.240 |
| Metastasis Carcinoma (N = 10) | 1141 | 10590 | 0.076 |
| Ovary Carcinoma (N = 20) | 127 | 1545 | 0.07 |
| Colon Rectal Carcinoma (N = 20) | 102 | 1875 | 0.034 |
| Fibrosis (N = 69) | 68 | 2221 | 0.038 |
| Apparently Healthy Blood Donor | 37 | 570 | 0.069 |
| (N = 120) | | | |

As is evident from the table and FIG. 1, individuals having a high ratio of NT-proBNP/GDF-15 suffer from a cardiac disorder, whereas individuals having a ration of <0.12 suffer from a different disorder.

What is claimed is:

1. A method for diagnosing a cardiac disorder in a subject, the method comprising
providing a sample from the subject,
determining an amount of a natriuretic peptide in the sample,
determining an amount of growth differentiation factor 15 (GDF-15) in the sample, and
calculating a ratio of natriuretic peptide to GDF-15 from the amounts determined, wherein a diagnosis of cardiac disorder in the subject is made based on the ratio calculated.

2. The method of claim 1, wherein a ratio of ≧0.10 is indicative of a cardiac disorder.

3. The method of claim 1, wherein a ratio of ≧0.12 is indicative of a cardiac disorder.

4. The method of claim 1, wherein a ratio of ≧0.15 is indicative of a cardiac disorder.

5. The method of claim 1, wherein the cardiac disorder is myocardial dysfunction, with or without heart failure, or acute coronary syndrome.

6. The method of claim 1, wherein the cardiac disorder is heart failure.

7. The method of claim 6, wherein the heart failure is symptomless.

8. The method of claim 1, wherein the natriuretic peptide is selected from the group consisting of brain natriuretic peptide (BNP), N-terminal pro brain natriuretic peptide (NT-proBNP), atrial natriuretic peptide (ANP), and N-terminal pro atrial natriuretic peptide (NT-proANP).

9. The method of claim 8, wherein the natriuretic peptide is NT-proBNP.

10. The method of claim 1, wherein a ratio of <0.1 is indicative of a disease other than cardiac disorder.

11. A device for diagnosing a cardiac disorder in a subject exhibiting atrial fibrillation, the device comprising
a means for determining an amount of a natriuretic peptide in a sample from the subject,
a means for determining an amount of growth differentiation factor 15 (GDF-15) in the sample from the subject,
optionally a means for calculating a ratio of natriuretic peptide to GDF-15 from the amounts determined, and
optionally a means for diagnosing a cardiac disorder in the subject from the ratio calculated.

12. A kit adapted for carrying out the method of claim 1, wherein the kit comprises
instructions for carrying out the method,
components for determining an amount of a natriuretic peptide in a sample from the subject,
components for determining an amount of growth differentiation factor 15 (GDF-15) in a sample from the subject,
optionally a means for calculating a ratio of natriuretic peptide to GDF-15 from the amounts determined, and
optionally a means for diagnosing a cardiac disorder in the subject from the ratio calculated.
